(19) United States
(12) Patent Application Publication
(10) Pub. No.: US 2013/0045885 A1
(43) Pub. Date: Feb. 21, 2013

(54) MATERIALS AND METHODS FOR PROFILING MICRORNAS

(71) Applicants: Shyam S. Mohapatra, Lutz, FL (US); Jia-Wang Wang, Tampa, FL (US)

(72) Inventors: Shyam S. Mohapatra, Lutz, FL (US); Jia-Wang Wang, Tampa, FL (US)

(73) Assignee: Univeristy of South Florida, Tampa, FL (US)

(21) Appl. No.: 13/579,316

(22) PCT Filed: Feb. 18, 2011
(86) PCT No.: PCT/US11/25532

§ 371 Date: Oct. 30, 2012

Related U.S. Application Data
(60) Provisional application No. 61/305,917, filed on Feb. 18, 2010.
(60) Provisional application No. 61/306,318, filed on Feb. 19, 2010.

(51) Int. Cl. C40B 30/04; C12Q 1/68; C07H 21/04

(52) U.S. Cl. 506/9; 536/24.33; 536/24.31; 435/6.12; 435/6.11

(57) ABSTRACT

The present invention provides materials and methods for detecting, quantifying, and/or profiling microRNAs. Advantageously, the present invention is sensitive, specific, convenient, and cost-effective. In one embodiment, the present invention provides a universal primer for reverse transcription of miRNAs, a universal reverse primer for PCR amplification reaction, and a universal probe. In another embodiment, the present invention provides assays that allow the detection and/or quantification of a plurality of target miRNAs using a single reverse transcription reaction and a single qPCR reaction.

### CROSS-REFERENCE TO RELATED APPLICATIONS

The present application claims the benefit of U.S. Provisional Application Ser. No. 61/305,917, filed Feb. 18, 2010, and U.S. Provisional Application Ser. No. 61/306,318, filed Feb. 19, 2010, each of which is hereby incorporated by reference herein in its entirety, including figures, tables, nucleic acid sequences, amino acid sequences, and drawings.

### BACKGROUND OF THE INVENTION

MicroRNAs (miRNAs), typically 18 to 25 nt in length, are non-protein-coding RNAs that can inhibit the translation of target mRNAs (Croce and Calin. 2005. miRNAs, cancer, and stem cell division. Cell 122(1): 6-7). miRNAs directly or indirectly regulate a wide range of genes, and are involved in a remarkable spectrum of biological pathways including cell development, proliferation and apoptosis (He and Hannon. 2004. MicroRNAs: small RNAs with a big role in gene regulation. Nat Rev Genet 5(7): 522-31, Alvarez-Garcia and Miska. 2005. MicroRNA functions in animal development and human disease. Development 132(21): 4653-62). As of September 2009, 10883 miRNA entries from vertebrates, flies, worms, plants, and viruses, including 721 human miRNAs and 579 mouse miRNAs, have been annotated (miRBase, Release 14) in the Sanger Institute miRNA sequence database (Griffiths-Jones, Saini, van Dongen and Enright. 2008. miRBase: tools for microRNA genomics. Nucleic Acids Res 36(Database issue): D154-8); the function of many miRNAs is unknown.

Materials and methods that can detect and quantify miRNAs with high sensitivity and specificity are useful. Cellular miRNA profile can offer insight in gene expression, and allow the determination of the species, tissue types, and developmental stages of a given tissue sample. Further, the detection and quantification of miRNAs may lead to the discovery of novel, miRNA-based diagnostic/prognostic biomarkers and therapeutic agents.

Conventional techniques for miRNA profiling include Northern hybridization, cloning, and microarray analysis. (Wang, Ach and Curry. 2007. Direct and sensitive miRNA profiling from low-input total RNA. RNA 13(1): 151-9, Wang and Cheng. 2008. A simple method for profiling miRNA expression. Methods Mol Biol 414: 183-90, Shingara, Keiger, Shelton, Laosinchai-Wolf, Powers, Conrad, Brown and Labourier. 2005. An optimized isolation and labeling platform for accurate microRNA expression profiling. RNA 11(9): 1461-70, Nelson, Baldwin, Scearce, Oberholtzer, Tobias and Mourelatos. 2004. Microarray-based, high-throughput gene expression profiling of microRNAs. Nat Methods 1(2): 155-61). These techniques are not as sensitive or specific, when compared to quantitative real-time reverse transcription PCR (qRT-PCR).

Several qRT-PCR-based methods have been developed for detecting and quantifying miRNAs (Li, Yao, Huang, Wang, Sun, Fan, Chang, Li, Wang and Xi. 2009. Real-time polymerase chain reaction microRNA detection based on enzymatic stem-loop probes ligation. Anal Chem 81(13): 5446-51, Varkonyi-Gasic, Wu, Wood, Walton and Hellens. 2007. Protocol: a highly sensitive RT-PCR method for detection and quantification of microRNAs. Plant Methods 3: 12, Ro, Park, Jin, Sanders and Yan. 2006. A PCR-based method for detection and quantification of small RNAs. Biochem Biophys Res Commun 351(3): 756-63).

The most frequently used qRT-PCR-based method, developed by Chen et al. (Chen, Ridzon, Broomer, Zhou, Lee, Nguyen, Barbisin, Xu, Mahuvakar, Andersen, Lao, Livak and Guegler. 2005. Real-time quantification of microRNAs by stem-loop RT-PCR. Nucleic Acids Res 33(20): e179), includes two main steps: reverse transcription of miRNAs using stem-loop RT primers, followed by a TaqMan® PCR analysis.

However, the Chen et al. method has several limitations. According to Chen et al., profiling each target miRNA requires a target-specific TaqMan® probe and a target-specific RT primer. As a result, the cost of making hundreds of target-specific probes and RT primers during miRNA screening tests can be prohibitive. In addition, the Chen et al. method is procedurally complex. Profiling each miRNA requires an RT reaction; otherwise, if only one RT reaction is performed, all miRNA-specific RT primers need to be mixed together. Further, hundreds of target-miRNA specific TaqMan® probes need to be added separately in order to detect or quantify miRNA. Moreover, RT primers used in the Chen et al. method only have a 6-nt-sequence that base-pairs with the target miRNAs. As a result, the RT primers may hybridize to, and prime other RNAs during RT reaction (Tang, Hajkova, Barton, Lao and Surani. 2006. MicroRNA expression profiling of single whole embryonic stem cells. Nucleic Acids Res 34(2): e9). Accordingly, improved methods for profiling miRNAs are needed.

### BRIEF SUMMARY OF THE INVENTION

The present invention provides materials and methods for detecting, quantifying, and/or profiling microRNAs. Advantageously, the present invention is sensitive, specific, convenient, and cost-effective.

In one aspect, materials for detecting, quantifying, and/or profiling microRNAs comprise: a universal primer for reverse transcription of miRNAs, a universal reverse primer for PCR amplification reaction, and a universal probe. Also provided are reagents and kits for detecting, quantifying, and/or profiling miRNAs.

In some embodiments, the universal primer for reverse transcription is an oligonucleotide comprising: a (dT)ₙ sequence flanked by a stem-looped universal adaptor sequence, wherein “n” is an integer ranging from 10 to 50, wherein the universal primer comprises at least two nucleotides adjacent to the 3′end of the (dT)ₙ sequence, and the nucleotide immediately adjacent to the (dT)ₙ sequence is not T, and wherein the universal adaptor sequence near the 5′end of the (dT)ₙ sequence forms a stem-loop structure by base-pairing.

In one embodiment, the universal reverse primer is an oligonucleotide comprising a sequence that is, or base-pairs with, at least part of the adaptor sequence near the 5′end of the (dT)ₙ sequence. In one embodiment, the universal probe comprises a sequence that is, or base-pairs with, at least part of the adaptor sequence near the 5′end of the (dT)ₙ sequence.

In some embodiments, the universal primer for reverse transcription comprises SEQ ID NO: 1. In some embodiments, the universal reverse primer for PCR amplification comprises SEQ ID NO: 2. In some embodiments, the universal probe comprises SEQ ID NO: 3.

In another aspect, the present invention provides assays for detecting, quantifying, and/or profiling miRNAs. In one embodiment, the present invention can detect a plurality of target miRNAs using one reverse transcription reaction and one qPCR reaction. Advantageously, the present invention can detect, quantify, and/or profile miRNAs at a level of about 1 pg.

In one embodiment, the method for detecting, quantifying, and/or profiling a target miRNA comprises:

a) contacting a sample comprising miRNAs with an effective amount of poly(A)polymerase molecules to yield 3′ end-polyadenylated miRNA molecules;

b) contacting the sample with an effective amount of a universal primer for reverse transcription and reverse transcriptases, and reverse transcribing the polyadenylated miRNA molecules to yield corresponding c-DNA molecules; and

c) contacting the sample with a sufficient amount of a universal reverse primer and a forward primer, and amplifying the corresponding c-DNA molecules using an amplification reaction (e.g., PCR).

### BRIEF DESCRIPTION OF THE DRAWINGS

FIG. 1 is a schematic illustration of an embodiment of the qRT-PCR assay for profiling miRNA, using a universal TaqMan® probe and a universal RT primer. Briefly, miRNAs are polyadenylated using poly(A)polymerase molecules. The poly(A)-tailed miRNAs are then reverse-transcribed into cDNAs using a universal primer comprising oligo dTs flanked by a stem-loop adaptor sequence. Finally, the cDNAs are amplified by qRT-PCRs using a mature target miRNA sequence as the forward primer and a universal reverse primer (QRTU). In one embodiment, a universal TaqMan® probe is used to detect the amplification product. Q=Quencher, such as IABlk_FQ (Iowa Black™ FQ), F=Fluorescent Dye, such as FAM (6-carboxyfluorescein).

FIG. 2 shows characteristics of the universal probe and RT primer (UPR) miRNA real-time quantitative PCR (qRT-PCR) assay. (A) Amplification curve plot of miR-690 UPR qRT-PCR assay. (B) Relative standard curve plot of miR-690 UPR qRT-PCR assay calculated over 10 serial dilutions. Average C_{T} values (y-axis) are plotted against the logarithm of the input amount of RNA (x-axis) added to each sample. Standard curve of the slope was −3.4 and correlation coefficient was 0.9995. (C) Correlation of total RNA input to the threshold of cycle (CT) values in four miRNA assays. Mouse spleen total RNA input ranged from 10 ng to 0.1 pg per RT reaction. (D) Amplification curve plot of 96 miRNA amplicons in a UPR qRT-PCR assay. The experiments were conducted in triplicate. During QPCR, a forward primer specific to each target miRNA and a universal reverse primer (QRTU) were used.

FIG. 3 shows that the UPR Q-RT-PCR assay is miRNA-specific. (A) Amplification curve plot of the UPR miRNA qRT-PCR assay, using total RNA as the template for reverse transcription without the step of polyadenylation (mRNA). When mRNAs are used as templates for amplification of let-7, miR-21, miR-142, miR-150 and miR-494, the cycle threshold values (Cts) of qRT-PCRs are greater than 38. NTCs (nontemplate controls) did not produce any detectable signals. The Il-4 and Il-18 amplifications of the cDNA molecules serve as positive controls of the RT reaction. (B) Amplification curve plot of the UPR miRNA qRT-PCR assay for let-7, miR-21, miR-142, miR-150, miR-494 and miR-690. Mouse tail genomic DNA (DNA) and transcribed spleen total RNA (miRNA, served as positive controls) were subjected to polyadenylation and reverse transcription reactions. There are no detectable signals for the DNA template. (C) Amplification curve plot of UPR miRNA qRT-PCR assay using mouse tail genomic DNA as the template of the 96 miRNA qPCR array. (D) Agarose gel image of the 12 miRNA reactions from the 96-miRNA qPCR array assay. After 40 cycles, products were electrophoresed on 2% agarose gels. A 1 kb DNA marker (Invitrogen) was loaded on the left side of the gel.

FIG. 4 shows characteristics of the SYBR Green real-time quantitative PCR (qRT-PCR) assay for 96 miRNAs. The SYBR Green Q-PCRs were performed using SYBR Green. For each miRNA, a forward primer specific to the target miRNA and a universal reverse primer were used for qPCR amplification. (A) Dissociation curves of all 96 miRNA amplicons amplified using miRNA cDNAs prepared from mouse spleens. (B) Amplification curve plot of 96 miRNA amplicons of the SYBR Green Q-PCR assay. The experiments were conducted in triplicate.

### BRIEF DESCRIPTION OF THE SEQUENCES

SEQ ID NO: 1 is a universal primer sequence for reverse transcription of miRNAs (RTUloop).

SEQ ID NO: 2 is a universal reverse primer sequence in qPCR reaction (QRTU).

SEQ ID NO: 3 is a universal probe sequence (miRU probe).

SEQ ID NO: 4 is a forward primer sequence for qPCR reaction (Let-7a).

SEQ ID NO: 5 is a forward primer sequence for qPCR reaction (miR-21).

SEQ ID NO: 6 is a forward primer sequence for qPCR reaction (miR-142).

SEQ ID NO: 7 is a forward primer sequence for qPCR reaction (miR-150).

SEQ ID NO: 8 is a forward primer sequence for qPCR reaction (miR-494).

SEQ ID NO: 9 is a forward primer sequence for qPCR reaction (mmu-miR-690).

### DETAILED DISCLOSURE OF THE INVENTION

The present invention provides materials and methods for detecting, quantifying, and/or profiling microRNAs. The present invention uses a reverse transcription reaction and a PCR amplification reaction. In one embodiment, the present invention uses a universal probe, such as a TaqMan® probe, and a universal RT-primer (UPR).

Advantageously, the UPR qRT-PCR assay can be sensitive, specific, convenient, and cost-effective. In one embodiment, the present invention uses a universal probe (such as a TaqMan®) and a universal RT primer (UPR). In a preferred embodiment, the present invention can detect a plurality of target miRNAs using one RT reaction and a single universal probe. Advantageously, the present invention allows detection and quantification of miRNAs in as little as 1 pg total RNA. Further, genomic DNA and mRNA in total RNA samples produce no or little detectable signals.

Total RNA used in the present invention can be obtained from simple extraction methods, such as, Trizol extraction. Total RNA samples used in the present invention need not be treated with DNases or undergo small RNA fractionation or purification, which are not only labor intensive procedures, but also may result in significant loss of input miRNAs.

The miRNA UPR qRT-PCR assay of the present invention has two major advantages when compared to the conventional miRNA stem-loop qRT-PCR methods (such as the Chen et al. method). First, a highly specific universal poly (T) primer, for example with a stretch of 25Ts, is used to prime the RT reaction for detection of all target miRNAs. In contrast, the conventional methods use a stem-loop RT primer that has a 6 nt sequence specific to each target miRNA sequence. For example, the 25Ts poly (T) sequence in the universal primer theoretically appears only once in a random sequence of 1.1259E+15 bps, while the 6 nt-sequence in the miRNA-specific primer appears 652962 times in a random sequence of the mouse genomic size. Further, the 6 nt, target-miRNA-specific primer used in the prior art methods is not genome-wide specific; It can not only prime the target miRNAs, but also prime other RNAs that have the 6 nt sequences. Further, using the prior art primers requires lower temperature (16° C.) for RT reaction (Chen, Ridzon, Broomer, Zhou, Lee, Nguyen, Barbisin, Xu, Mahuvakar, Andersen, Lao, Livak and Guegler. 2005. Real-time quantification of microRNAs by stem-loop RT-PCR. Nucleic Acids Res 33(20): e179). Therefore, the present invention can greatly increase the priming specificity during reverse transcription, since only RNAs that have poly (A) tails can be reversely transcribed. The decrease of non-specificity in the RT reaction increases the sensitivity of the qRT-PCR assay.

Moreover, the present invention can use a universal probe (such as a universal TaqMan® probe) for the detection and qualification of a plurality of miRNAs. In contrast, the conventional methods require target-miRNA-specific TaqMan® probes, where each probe can only detect one target miRNA. In addition, conventional methods require that each target-specific probe need to be individually added each time.

Materials for miRNA Profiling

One aspect of the present invention provides materials for detecting, quantifying, and/or profiling miRNAs. In one embodiment, the materials comprise: a universal primer for reverse transcription of miRNAs, a universal reverse primer for PCR amplification reaction, and a universal probe. Also provided are reagents and kits for detecting, quantifying, and/or profiling miRNAs.

Design of Universal Primers for Reverse Transcription of miRNAs

In one aspect, the present invention provides a universal primer for reverse transcription of miRNAs. In some embodiments, the universal primer is an oligonucleotide sequence comprising: a (dT)ₙ sequence flanked by a stem-looped universal adaptor sequence, wherein n is an integer ranges from 10 to 50, wherein the universal primer comprises at least two nucleotides adjacent to the 3′end of the (dT)ₙ sequence, and the nucleotide immediately adjacent to the (dT)ₙ sequence is not T, and wherein the universal adaptor sequence near the 5′end of the (dT)ₙ sequence forms into a stem-loop structure by base-pairing. In one embodiment, the universal primer for reverse transcription is single-stranded DNA.

In some embodiments, n is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. In some embodiments, n can be an integer less than 10 or greater than 50.

In some embodiments, the universal primer comprises at least 2, 3, 4, 5, 6, 7, 8, 9, or nucleotides are immediately adjacent to the 3′end of the (dT)ₙ sequence.

In some embodiments, the adaptor sequence comprises 20, 21, 22, 23, 24, 25, 26, 27, 28, 29, 30, 31, 32, 33, 34, 35, 36, 37, 38, 39, 40, 41, 42; 43, 44, 45, 46, 47, 48, 49, 50, 51, 52, 53, 54, 56, 57, 58, 59, or 60 nucleotides. In some embodiments, the adaptor sequence comprises more than 60 nucleotides. In some embodiments, each stem of the adaptor sequence located near the 5′end of the (dT)ₙ sequence comprises 10, 11, 12, 13, 14, 15, 16, 17, 18, 19, 20, 21, 22, 23, 24, or 25 nucleotides. In some embodiments, each stem of the adaptor sequence located near the 5′end of the (dT)ₙ sequence comprises more than 25 nucleotides. In some embodiments, the loop of the adaptor sequence comprises 10, 11, 12, 13, 14, 15, 16, 17, 18, 19, 20, 21, 22, 23, 24, or 25 nucleotides. In certain embodiments, the loop of the adaptor sequence can comprise more than 25 nucleotides.

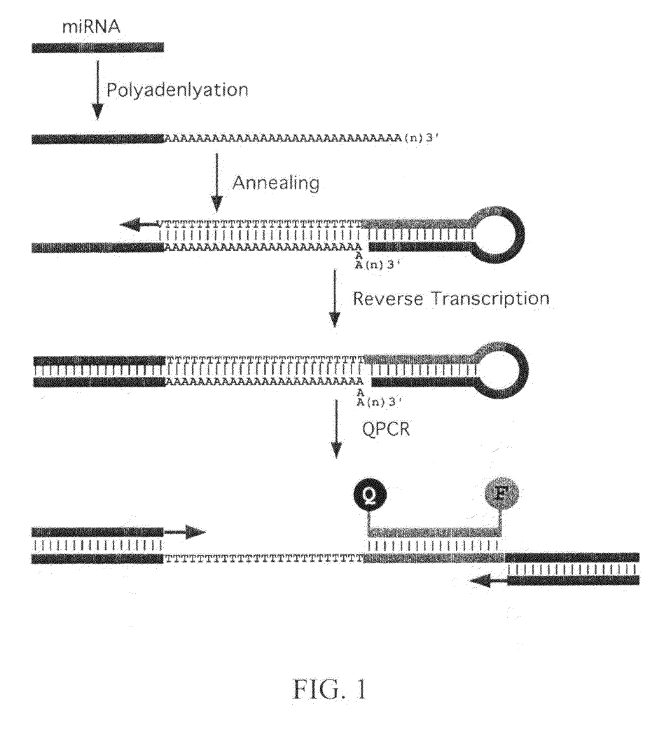

FIG. 1 illustrates an embodiment of the universal primer for reverse transcription of miRNAs. In a specific embodiment, the universal primer for reverse transcription of miRNAs comprises SEQ ID NO: 1.

In some embodiments, the adaptor sequence (near the ′5 end) of the universal primer for reverse transcription of miRNAs does not comprise a sequence that hybridizes, or base-pairs, with the target miRNA, wherein the sequence is at least 2, 3, 4, 5, 6, 7, 8, 9, 10, 11, 12, 13, 14, 15, 16, 17, 18, 20 nucleotides in length.

### Design of Universal Reverse Primers for PCR Amplification Reaction

In one aspect, the present invention provides a universal reverse primer for PCR amplification reaction. In some embodiments, the universal reverse primer is an oligonucleotide comprising a sequence that is, or base-pairs with, at least part of the adaptor sequence (located near the 5′end of the (dT)ₙ sequence) of the universal primer for reverse transcription. In some embodiments, the universal reverse primer is single-stranded DNA.

In some embodiments, the universal reverse primer for PCR amplification comprises 10, 11, 12, 13, 14, 15, 16, 17, 18, 19, 20, 21, 22, 23, 24, or 25 nucleotides. In some embodiments, the universal reverse primer for PCR amplification comprises more than 25 nucleotides.

FIG. 1 illustrates an embodiment of the universal reverse primer for PCR amplification reaction. In a specific embodiment, the universal reverse primer for PCR amplification reaction comprises SEQ ID NO: 2.

In certain embodiments, the universal reverse primer for PCR amplification does not comprise a sequence that is, or base-pairs with, the target miRNA sequence.

### Design of Forward Primers for PCR Amplification Reaction

In one aspect, the present invention provides a forward primer for PCR amplification reaction. In some embodiments, the forward primer is an oligonucleotide comprising a sequence that hybridizes, or base-pairs with, at least part of the target miRNA sequence. In some embodiments, the forward primer is single-stranded DNA.

The forward primer can be single-stranded DNA or RNA. In some embodiments, the forward primer comprises a sequence that hybridizes, or base-pairs with, the entire target miRNA sequence. In some embodiments, the forward primer comprises a sequence that hybridizes, or base-pairs with, part of the target miRNA sequence. In certain embodiments, the sequence that hybridizes, or base-pairs with, the entire miRNA sequence has 6, 7, 8, 9, 10, 11, 12, 13, 14, 15, 16, 17, 18, 19, 20, 21, 22, 23, 24, or 25 nucleotides. In certain embodiments, the forward primer has 6, 7, 8, 9, 10, 11, 12, 13, 14, 15, 16, 17, 18, 19, 20, 21, 22, 23, 24, or 25 nucleotides. In certain embodiments, the forward primer can comprise more than 25 nucleotides.

FIG. 1 illustrates an embodiment of the forward primer for PCR amplification reaction.

### Design of Universal Probes

In one aspect, the present invention provides a universal probe. The universal probes are useful for the detection and/or quantification of target miRNAs. In some embodiments, the universal probe is an oligonucleotide comprising a sequence that is, or base-pairs with, at least part of the adaptor sequence of the universal primer for reverse transcription. In some embodiments, the universal probe is single-stranded DNA.

In some embodiments, the universal probe further comprises a fluorophore, or other detectable moiety, attached at the ends of the oligonucleotide.

In certain embodiments, the universal probe in PCR amplification comprises 10, 11, 12, 13, 14, 15, 16, 17, 18, 19, 20, 21, 22, 23, 24, or 25 nucleotides. In certain embodiments, the universal probe can comprise more than 25 nucleotides.

FIG. 1 illustrates an embodiment of the universal probe. In a specific embodiment, the universal probe comprises SEQ ID NO: 3.

In certain embodiments, the universal probe does not comprise a sequence that is, or base-pairs with, the target miRNA sequence.

The detectable moiety is preferably a fluorophore. The fluorophores are preferably attached to the ends or near the ends of the oligonucleotide. When a probe of the invention hybridizes with a target nucleic acid sequence, the probe undergoes a conformational change to bring the fluorophores closer in proximity to each other. This change in distance causes a change in the photon absorption or emission of the fluorophores, creating a visual indication that the probe of the invention has bound a target sequence.

Fluorescent resonance energy transfer (FRET) or non-FRET interactions are used to detect the binding of the probe to its target sequence (e.g., PCR amplification products). FRET interactions (also known as non-radiative energy transfer; see Yaron et al., Analytical Biochemistry 95:228-235 (1979)) for quenching fluorescence signals requires spectral overlap between the donor and acceptor fluorophore moieties and the efficiency of quenching is directly proportional to the distance between the donor and acceptor moieties of the FRET pair. Extensive reviews of the FRET phenomenon are described in Clegg, R. M., Methods Enzymol., 221: 353-388 (1992) and Selvin, P. R., Methods Enzymol., 246: 300-334 (1995). In contrast, non-FRET interactions (also known as radiationless energy transfer; See: Yaron et al., Analytical Biochemistry 95:228-235 (1979)) requires short range interaction by “collision” or “contact” between the fluorophore moieties and therefore requires no spectral overlap between the donor and acceptor pair.

When the probe binds to the target sequence, the probe will undergo a conformational change causing the distance and/or angle between the fluorophore pairs to change. This change can then be detected because it will change the efficiency of resonance energy transfer between the fluorophore moieties after exposure of the probe to an excitation wave-length of light.

In one embodiment, fluorophores useful according to the present invention include, but are not limited to, FAM (6-carboxyfluorescein), CY5, CY3, BODIPY FL, and TEXAS RED. In a preferred embodiment, the fluorophore is FAM.

The universal primer, probe and adaptor sequences can be derived from universal probe and primer sequences known in the art, such as universal TaqMan® probe and primer sequences. For example, the design of the stem-loop universal adaptor sequence is described in (Chen, Ridzon, Broomer, Zhou, Lee, Nguyen, Barbisin, Xu, Mahuvakar, Andersen, Lao, Livak and Guegler. 2005. Real-time quantification of microRNAs by stem-loop RT-PCR. Nucleic Acids Res 33(20): e179).

The oligonucleotides of the present invention can encompass single and double-stranded RNA, single and double-stranded DNA and cDNA, nucleic acid analogs, aptamers, and the like. The terms “nucleic acid” and “oligonucleotide” are used interchangeably herein. Preferably, the oligonucleotide strands of the probe are single-stranded DNA.

“Hybridization” refers to a reaction in which one or more polynucleotides react to form a complex that is stabilized via hydrogen bonding between a particular purine and a particular pyrimidine in double-stranded nucleic acid molecules (DNA-DNA, DNA-RNA, or RNA-RNA). The major specific pairings are guanine with cytosine and adenine with thymine or uracil. Various degrees of stringency of hybridization can be employed. The more severe the conditions, the greater the complementarity that is required for duplex formation. Severity of conditions can be controlled by temperature, probe concentration, probe length, ionic strength, time, and the like.

Preferably, hybridization is conducted under high stringency conditions by techniques well known in the art, as described, for example, in Keller, G. H. & M. M. Manak, DNA Probes, and the companion volume DNA Probes: Background, Applications, Procedures (various editions, including 2^{nd} Edition, Nature Publishing Group, 1993). Hybridization is also described extensively in the Molecular Cloning manuals published by Cold Spring Harbor Laboratory Press, including Sambrook & Russell, Molecular Cloning: A Laboratory Manual (2001). Each of these publications is incorporated herein by reference in its entirety.

A non-limiting example of high stringency conditions for hybridization is at least about 6×SSC and 1% SDS at 65° C., with a first wash for 10 minutes at about 42° C. with about 20% (v/v) formamide in 0.1×SSC, and with a subsequent wash with 0.2×SSC and 0.1% SDS at 65° C. A non-limiting example of hybridization conditions are conditions selected to be about 1, 2, 3, 4, 5, 6, 7, 8, 9, 10, 11, 12, 13, 14, 15, 16, 17, 18, 19, 20, 21, 22, 23, 24, or 25° C. lower than the thermal melting point (Tₘ) for the specific sequence in the particular solution. Tₘ is the temperature (dependent upon ionic strength and pH) at which 50% of the target sequence hybridizes to a perfectly matched probe. Tₘ typically increases with [Na⁺] concentration because the sodium cations electrostatically shield the anionic phosphate groups of the nucleotides and minimize their repulsion. The washes employed may be for about 5, 10, 15, 20, 25, 30, or more minutes each, and may be of increasing stringency if desired.

Calculations for estimating Tₘ are well-known in the art. For example, the melting temperature may be described by the following formula (Beltz, G. A., K. A. Jacobs, T. H. Eickbush, P. T. Cherbas, and F. C. Kafatos, Methods of Enzymology, R. Wu, L. Grossman and K. Moldave [eds.] Academic Press, New York 100:266-285, 1983).

Tm=81.5° C.+16.6 Log [Na+]+0.41(%G+C)−0.61(% formamide)−600/length of duplex in base pairs.

A more accurate estimation of Tₘ may be obtained using nearest-neighbor models. Breslauer, et al., Proc. Natl. Acad. Sci. USA, 83:3746-3750 (1986); SantaLucia, Proc. Natl Acad. Sci. USA, 95: 1460-1465 (1998); Allawi & SantaLucia, Biochemistry 36:10581-94 (1997); Sugimoto et al., Nucleic Acids Res., 24:4501-4505 (1996). Tₘ may also be routinely measured by differential scanning calorimetry (Duguid et al., Biophys J, 71:3350-60, 1996) in a chosen solution, or by other methods known in the art, such as UV-monitored melting. As the stringency of the hydridization conditions is increased, higher degrees of homology are obtained.

### Kits

The present invention also provides kits for detecting, quantifying, and/or profiling miRNA. In one embodiment, the kit comprises a universal primer for reverse transcription, a universal reverse primer for an amplification reaction (e.g., PCR), and/or a universal probe. Optionally, the kit can further comprise a forward primer sequence for an amplification reaction (e.g., PCR).

In a specific embodiment, the kit comprises SEQ ID NO:1 and/or SEQ ID NO:2. In another specific embodiment, the kit further comprises SEQ ID NO:3. In a further specific embodiment, the kit comprises one or more of SEQ ID NOs: 4-9.

Optionally, the kit may include any material useful for performing any step of the present invention. For instance, the kit may further comprise any material useful for reverse transcription of RNAs, and/or for profiling the target RNA. For instance, the kit may poly(A)polymerases, dNTPs, Adenosine-5′-triphosphates (ATP), DNA ligases (e.g., T4 DNA ligase), and Taq DNA polymerase.

The kit may also comprise, e.g., a buffering agent, a preservative, or a stabilizing agent. Each component of the kit is usually enclosed within an individual container and all of the various containers are within a single package along with instructions (e.g., printed instructions).

UPR qRT-PCR Assay

Another aspect of the present invention provides an assay for detecting, quantifying, and/or profiling miRNAs. Advantageously, the present invention can detect, quantify, and/or profile miRNAs in a sample of about 1 pg.

In one embodiment, the method for detecting, quantifying, and/or profiling a target miRNA comprises:

a) contacting a sample containing miRNAs with an effective amount of poly(A)polymerase molecules to yield 3′ end-polyadenylated miRNA molecules,

b) contacting the sample with an effective amount of a universal primer for reverse transcription and reverse transcriptases, and reverse transcribing the polyadenylated miRNA molecules to yield corresponding c-DNA molecules, and

c) contacting the sample with an effective amount of a universal reverse primer and a forward primer, and amplifying the corresponding c-DNA molecules using an amplification reaction (e.g., PCR);

wherein the universal primer for reverse transcription is an oligonucleotide comprising: a (dT)ₙ sequence flanked by a stem-looped universal adaptor sequence, wherein n is an integer ranges from 10 to 50, wherein the universal primer comprises at least two nucleotides adjacent to the 3′end of the (dT)ₙ sequence, and the nucleotide immediately adjacent to the (dT)ₙ sequence is not T, and wherein the universal adaptor sequence near the 5′end of the (dT)ₙ sequence forms into a stem-loop structure by base-paring,

wherein the universal reverse primer for the amplification reaction is an oligonucleotide comprising a sequence that is, or base-pairs with, at least part of the adaptor sequence of the universal primer for reverse transcription, and

wherein the forward primer for the amplification reaction is an oligonucleotide comprising a sequence that hybridizes, or base-pairs with, at least part of the target miRNA sequence.

In a further embodiment, the assay comprises the steps of:

contacting the sample with an effective amount of a universal probe,

detecting and/or quantifying a level of amplified c-DNA molecules, and

determining the level of the target miRNA based on the c-DNA level.

In one embodiment, the universal probe comprises a detectable moiety, for example, a fluorophore. In one embodiment, a plurality of detectable moieties (e.g., fluorophores) can be used.

In one embodiment, the amplification reaction (e.g., PCR) for amplifying cDNAs is quantitative real time polymerase chain reaction (qRT-PCR).

In one embodiment, the PCR reaction is performed once. In another embodiment, the reverse transcription reaction is performed once.

In one embodiment, the miRNA profiling assay of the present invention uses one universal primer for reverse transcription, one universal reverse primer in the amplification reaction (e.g., PCR), and/or one universal probe. In one embodiment, the miRNA profiling assay detects, quantifies, and/or profiles a plurality of target miRNAs.

In one embodiment, the threshold cycle (Ct) of the PCR amplification reaction ranges from 15 to 37. In preferred embodiments, the Ct value of the PCR amplification reaction ranges from 17 to 35, 20 to 30, 23 to 33, or 25 to 30. In certain embodiments, the Ct value is 15, 16, 17, 18, 19, 20, 21, 22, 23, 24, 25, 26, 27, 28, 29, 30, 31, 32, 33, 34, 35, 36, or 37. In one embodiment, the Ct value does not exceed 38.

Alternatively, the UPR qRT-PCR assay can be used for detecting, quantifying, and/or profiling nucleotide molecules, such as for example, siRNAs, oligonucleotides, polynucleotides, and/or mRNAs.

In one embodiment, samples containing miRNAs is a total RNA sample. In certain embodiments, the sample contains DNA, or has not been treated with DNases, or both. The samples can be derived from an organism, including mammals such as apes, chimpanzees, orangutans, humans, monkeys; and domesticated and/or laboratory animals such as dogs, cats, horses, cattle, pigs, sheep, goats, chickens, mice, rats, guinea pigs, and hamsters. The samples can be derived from, including but not limited to, a sample containing tissues, cells, and/or biological fluids isolated from a subject.

### Materials and Methods

Table 1 illustrates oligonucleotide sequences useful according to the present invention.

TABLE 1

| Name | Sequence (5′ to 3′) |
|---|---|
| RTUloop | TGGCTAGTTAAGCTCACCAGCTCGGTACCAAGCTTAACT |
| | AGCCA(T)25VN* (SEQ ID NO: 1) |
| QRTU | TGGCTAGTTAAGCTCACCAGCTCG (SEQ ID NO: 2) |
| miRU probe | FAM/TGGCTAGTTAAGCTTGGTACCGAGCT/IABlk_FQ |
| | (SEQ ID NO: 3) |
| Let-7a | TGAGGTAGTAGGTTGTATAGTT (SEQ ID NO: 4) |
| miR-21 | TAGCTTATCAGACTGATGTTGA (SEQ ID NO: 5) |
| miR-142 | TGTAGTGTTTCCTACTTTATGGA (SEQ ID NO: 6) |
| miR-150 | TCTCCCAACCCTTGTACCAGTG (SEQ ID NO: 7) |
| miR-494 | TGAAACATACACGGGAAACCTCTT (SEQ ID NO: 8) |
| mmu-miR-690 | AAAGGCTAGGCTCACAACCAAA (SEQ ID NO: 9) |

*V = A, G, or C; N = A, G, C, or T.

ABBREVIATIONS: miRNAs: MicroRNAs; UPR: a universal probe and RT primer; qRT-PCR: quantitative real-time reverse transcription PCR; QRTU: universe reverse primer; RTUloop: universal RT primer; FAM: 6-carboxyfluorescein; CT: threshold cycle.

Following are examples which illustrate procedures for practicing the invention. These examples should not be construed as limiting. All percentages are by weight and all solvent mixture proportions are by volume unless otherwise noted.

### Example 1

### The Universal Probe-RT Primer (UPR) Microrna Real-Time QRT-PCR Assay

FIG. 1 illustrates one embodiment of the universal probe-RT primer (UPR) real-time qRT-PCR assay for profiling microRNAs. The assay comprises a three-step process. First, total RNA is polyadenylated using poly(A)polymerase molecules. The polyadenylated miRNAs are reversely transcribed into cDNAs using a universal RT primer (RTUloop) with 25-T poly (T) at 3′ end. Finally, the RT product is amplified by qPCR using the mature target miRNA sequence as the forward primer and a universal reverse primer (QRTU). The amplification products can be detected or quantified using a miRU fluorescent probe, such as for example, a 26-nucleotide universal TaqMan® probe.

### MiRNA Sequences and Primers

About 800 human and mouse miRNA genes are selected from the Sanger Institute miRBase Sequence Database. miRNA expression profiling was performed and analyzed using the dot-blot array as previously described (Wang and Cheng. 2008. A simple method for profiling miRNA expression. Methods Mol Biol 414: 183-90). 96 miRNAs were selected for miRNA QPCR array assay from the dot blot array results.

The miRNA universal TaqMan® probe was designed by PrimerQuest (Table 1) based on sequences described in Ro et al. The stem-loop adaptor contained in the RT primers were designed according to Chen et al. (Chen, Ridzon, Broomer, Zhou, Lee, Nguyen, Barbisin, Xu, Mahuvakar, Andersen, Lao, Livak and Guegler. 2005. Real-time quantification of microRNAs by stem-loop RT-PCR. Nucleic Acids Res 33(20): e179). All DNA oligonucleotides were synthesized by IDT.

Preparation of RNA and cDNA

Total RNA was isolated from spleens of 4-week-old Balb/C mice using Trizol (Invitrogen) following the manufacturer's protocol. Total RNAs (1 μg) was incubated with poly(A)polymerase (USB) molecules, thereby generating polyadenylated microRNAs at the ′3 end. The polyadenylated RNAs (20 μl) reverse transcribed using 2 μM RTUloop primer, 0.25 mM each of dNTPs, 100 units Smartscribe reverse transcriptase, 1× reverse transcriptase buffer, and 10 mM DTT (Clontech Laboratories). The reactions were incubated at 42° C. for 90 minutes, and then at 95° C. for 5 minutes, to inactivate reverse transcriptase molecules and to degrade RNAs. All reverse transcriptase reactions included no-template and minus-RT as controls.

### Real-Time PCR

1 μl 10-time serially diluted reverse transcription reactions were used for real-time PCR, using the target miRNA as the forward primer and QRTU as the universal reverse primer. All the reactions were run on the ABI 7900HT System (Applied Biosystems). Real-time PCRs were carried out in a 20 μl reaction in triplicate.

Amplification curves were generated with an initial denaturing step at 95° C. for 30 seconds, followed by 40 cycles at 95° C. for 5 seconds, and at 60° C. for 30 seconds. Dissociation curves were generated using the following programs: PCR products were denatured at 95° C. for 15 seconds, cooled to 60° C. for 15 seconds, and finally at 95° C. for 15 seconds.

All QPCRs were carried out using Premix Ex Taq™ or SYBR Premix II Ex Taq (Perfect Real Time) (Clontech Laboratories). SYBR Green 1 and TaqMan® PCR products were visualized on 2% agarose gels by Grow-Green staining (eEnzyme).

### Example 2

### Reproducibility of the UPR Microrna QRT-PCR Assay

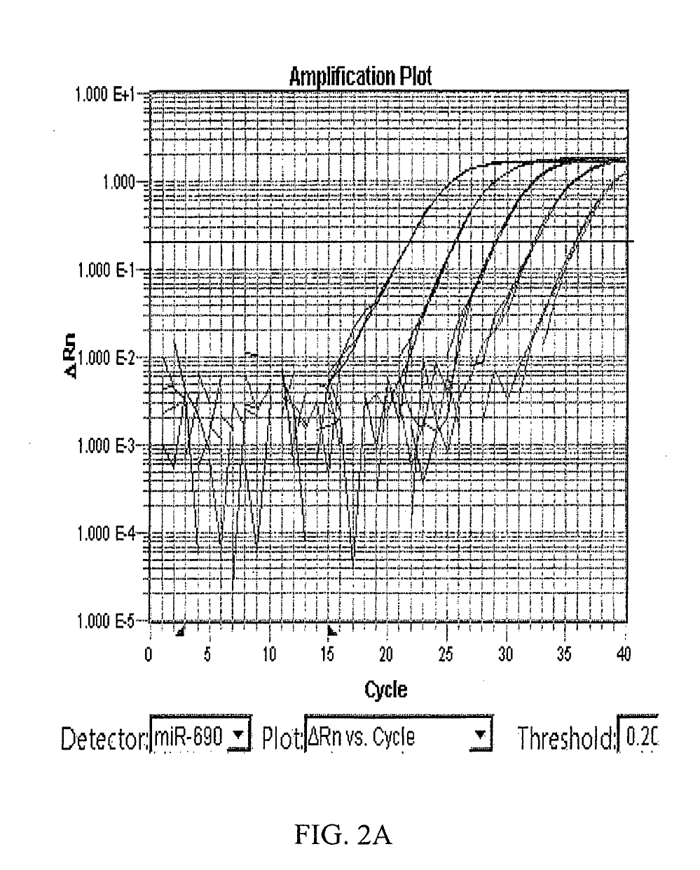

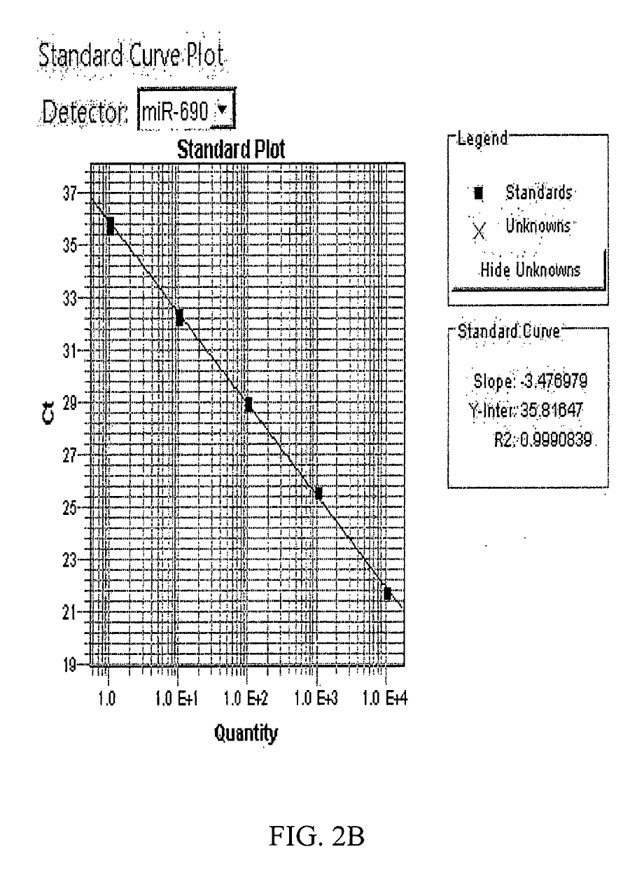

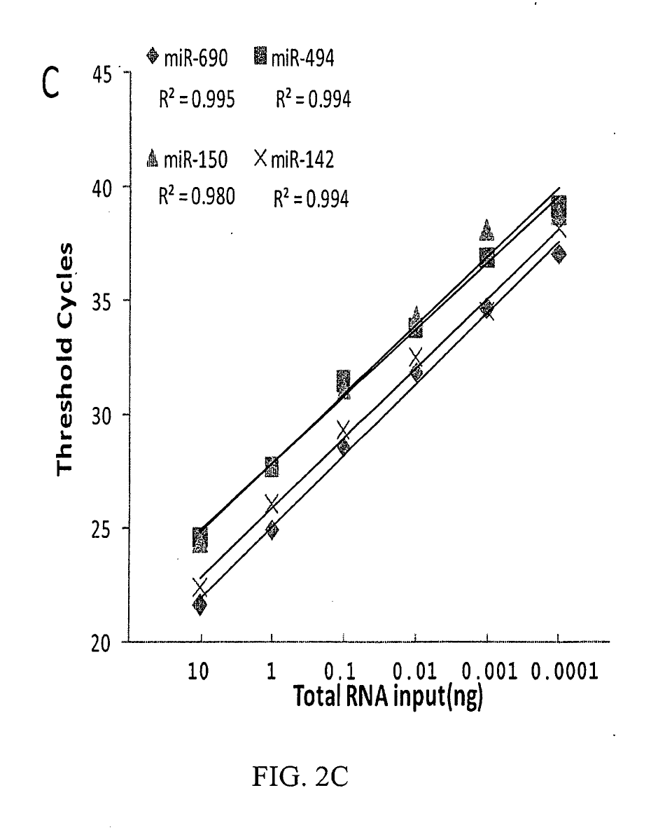

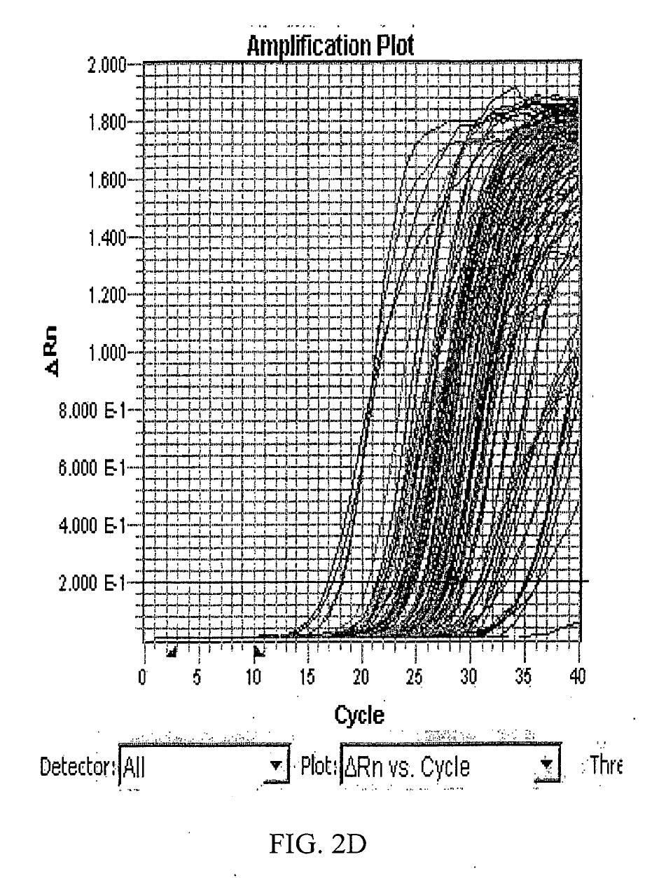

The amplification efficiency is critical a reproducible qRT-PCR assay. To examine the reproducibility of the UPR miRNA qRT-PCR assay, amplification efficiency was calculated using the relative standard curve method (FIGS. 2B&C). Briefly, qRT-PCR cDNA templates of miRNAs were prepared by 10-time serial dilution, which is equivalent to 10, 1, 0.1, 0.01 and 0.001 ng of total RNA, respectively. Each sample was run in triplicate.

The standard curves were generated using the Prism HT7900 system. The correlation coefficient (R2) values were greater than 0.99 (except for that of miR-150, which is 0.98), indicating excellent linear reliability between RNA concentration and the C_{T} value of reverse transcription real-time PCR reaction for each miRNA.

PCR efficiency was calculated by the formula E=−1+10^{-1/slop}. FIGS. 2A&B show the amplification curve plot and standard curve plot of the miR-690 qRT-PCR. The PCR efficiency is 0.94 for miR-690 and 1.1 for miR-142 and miR-150 (efficiencies between 0.90 and 1.10 are typically acceptable).

Amplification curves correlated to the concentration of RNA template and spanned five orders of magnitude (FIGS. 2A&B). The CT values from the amplification curves for the 96 miRNA QPCR array range from 17 to 35, equivalent to five orders of magnitude (FIG. 2D).

### Example 3

### Specificity of the UPR Microrna QRT-PCR Assay

Without the use of DNases, no RNA isolation method can consistently produce RNA free from genomic DNA contamination. This Example tests whether residual genomic DNA contamination may cause non-specificity of the UPR miRNA qRT-PCR assay.

Briefly, mouse tail genomic DNA, isolated by the DNeasy Blood & Tissue Kit (QiaGen), was used as the template in the UPR miRNA qRT-PCR assay. Mouse tail genomic DNA was added during the polyadenylation reaction (0.3 μg genomic DNA used), or QPCR reactions as templates (1 μg genomic DNA used).

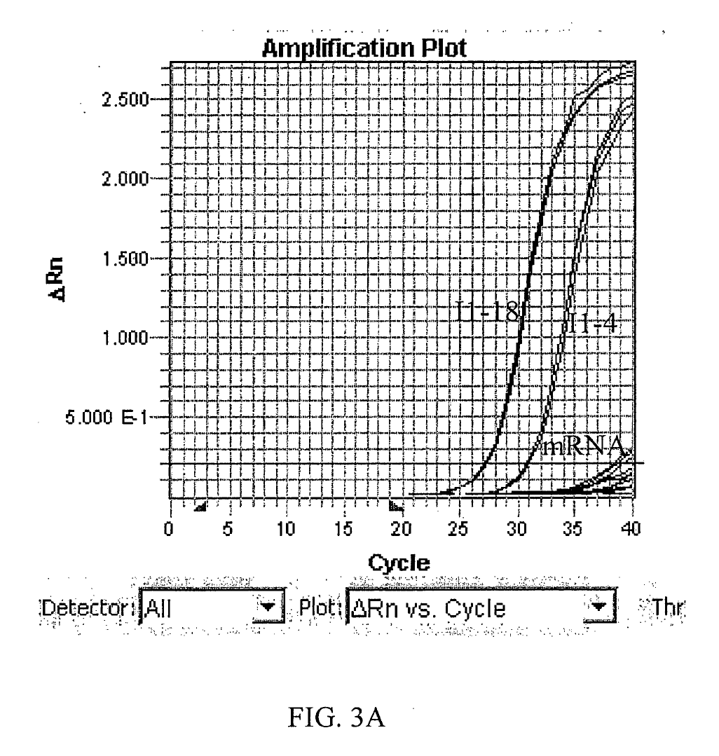

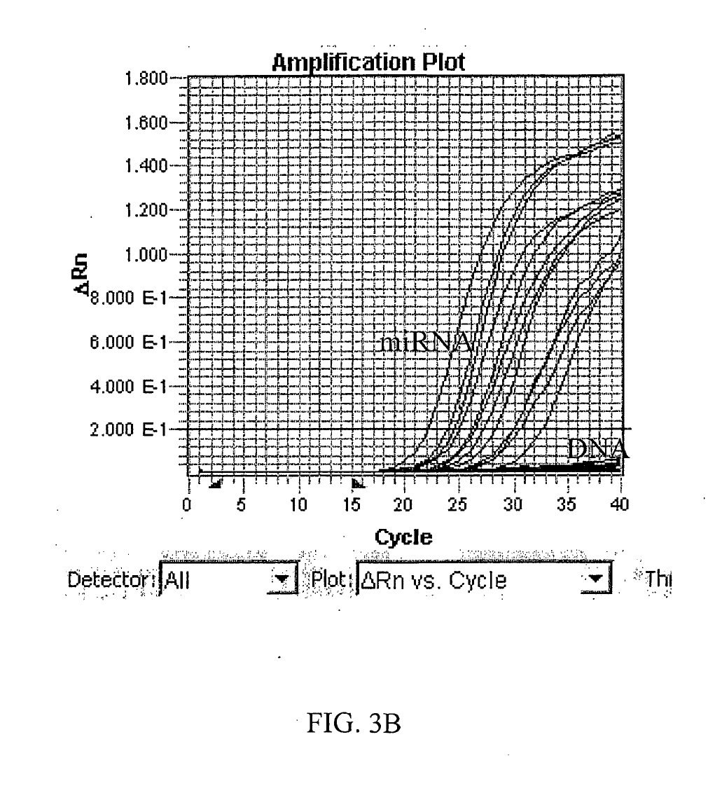

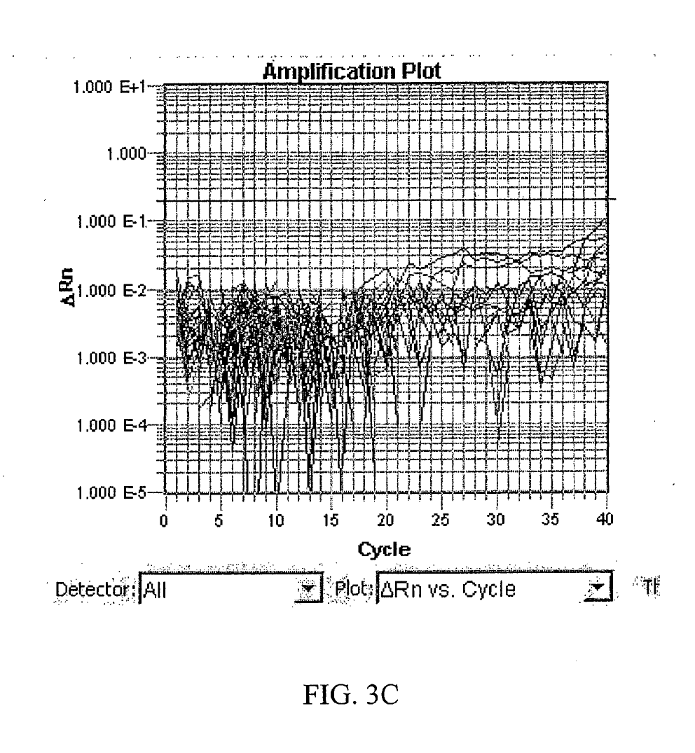

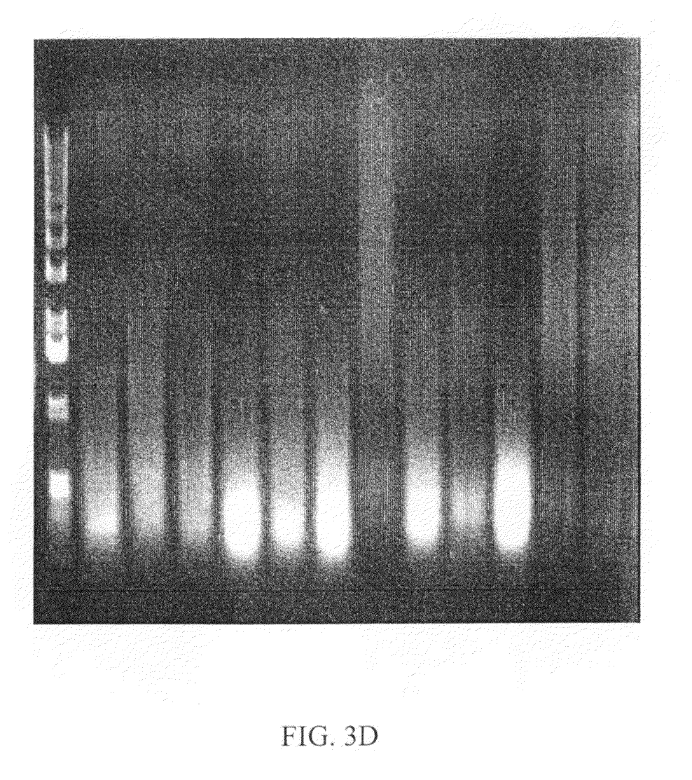

The results show no detectable signals in the UPR miRNA QPCR assay, when using genomic DNA either as a sham control for the total RNA (FIG. 3B), or as the direct template of QPCR (FIG. 3CC). The agarose gel electrophoresis shows nonspecific amplification (FIG. 3D), which may be detected by SYBR Green. The amount of the genomic DNA used in this Example far exceeds residual DNA that may be present in total RNA preparation. This indicates that the accuracy of the UPR miRNA qRT-PCR assay will not be affected by genomic DNA contamination in total RNA preparation.

Total RNA was also used as the template of UPR miRNA qRT-PCR assay without the polyadenylation step. This investigates whether mRNAs, the major component of total RNA, will produce significant non-specific signals in the assay. When polyadenylated mRNA is used as the template of the qRT-PCR assay, the CTs (threshold cycle) of qRT-PCR assays are greater than 38 (FIG. 3A). CT values greater than 35 approaches the sensitivity limits of the real-time PCR detection system of miRNAs. This suggests that the contribution of the background signals from mRNA in this assay is negligible.

### Example 4

### Detection of Non-Specific Amplification by Microrna SYBR Green QPCR Array

SYBR Green is a fluorescent dye. It non-specifically intercalates into double-stranded DNA and detects double-stranded DNA.

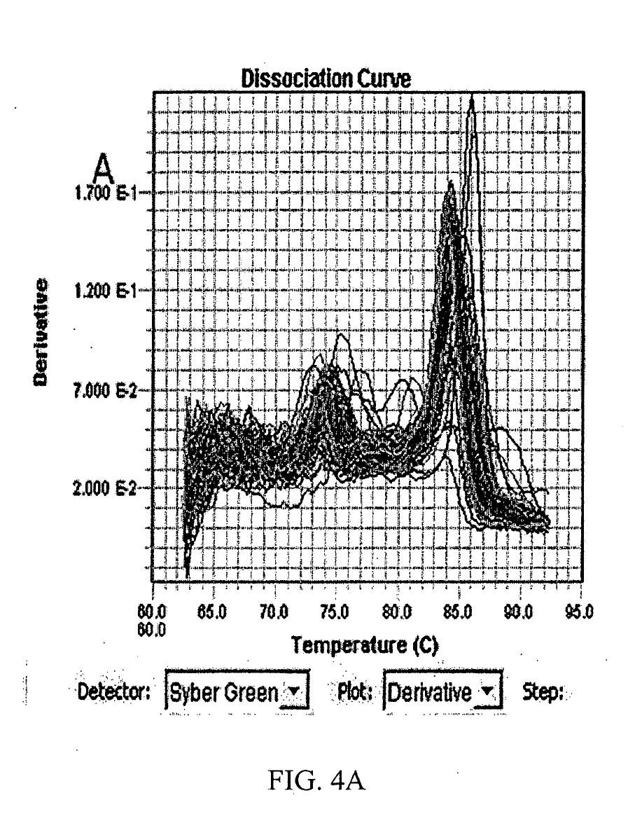

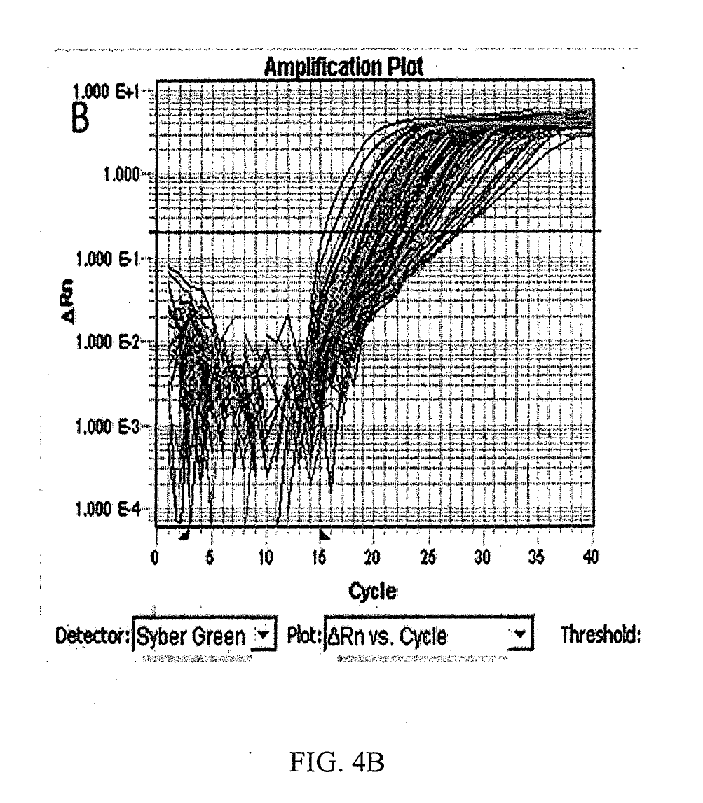

FIG. 4 shows detection of non-specific amplification by 96-miRNA qRT-PCR array using SYBR Green. The dissociation curves (FIG. 4A) show that most miRNA qRT-PCRs produced a small peak and a major peak. The small peak represents non-specific amplification, while the major peak represents amplification of the desired PCR products. The log amplification curve plot (FIG. 4B) shows that there are non-exponent amplifications for low abundant miRNAs. The results suggest that the SYBR Green assay may over-detect miRNAs, which is present in low amounts. It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

All patents, patent applications, provisional applications, and publications referred to or cited herein, supra or infra, are incorporated by reference in their entirety, including all figures and tables, to the extent they are not inconsistent with the explicit teachings of this specification.

### REFERENCES

C. M. Croce and G. A. Calin. 2005. miRNAs, cancer, and stem cell division. Cell 122(1): 6-7.
S. Griffiths-Jones, H. K. Saini, S. van Dongen and A. J. Enright. 2008. miRBase: tools for microRNA genomics. Nucleic Acids Res 36(Database issue): D154-8.
L. He and G. J. Hannon. 2004. MicroRNAs: small RNAs with a big role in gene regulation. Nat Rev Genet 5(7): 522-31.
I. Alvarez-Garcia and E. A. Miska. 2005. MicroRNA functions in animal development and human disease. Development 132(21): 4653-62.
H. Wang, R. A. Ach and B. Curry. 2007. Direct and sensitive miRNA profiling from low-input total RNA. RNA 13(1): 151-9.
J. W. Wang and J. Q. Cheng. 2008. A simple method for profiling miRNA expression. Methods Mol Biol 414: 183-90.
J. Shingara, K. Keiger, J. Shelton, W. Laosinchai-Wolf, P. Powers, R. Conrad, D. Brown and E. Labourier. 2005. An optimized isolation and labeling platform for accurate microRNA expression profiling. RNA 11(9): 1461-70.
P. T. Nelson, D. A. Baldwin, L. M. Scearce, J. C. Oberholtzer, J. W. Tobias and Z. Mourelatos. 2004. Microarray-based, high-throughput gene expression profiling of microRNAs. Nat Methods 1(2): 155-61.
J. Li, B. Yao, H. Huang, Z. Wang, C. Sun, Y. Fan, Q. Chang, S. Li, X. Wang and J. Xi. 2009. Real-time polymerase chain reaction microRNA detection based on enzymatic stem-loop probes ligation. Anal Chem 81(13): 5446-51.
E. Varkonyi-Gasic, R. Wu, M. Wood, E. F. Walton and R. P. Hellens. 2007. Protocol: a highly sensitive RT-PCR method for detection and quantification of microRNAs. Plant Methods 3: 12.
S. Ro, C. Park, J. Jin, K. M. Sanders and W. Yan. 2006. A PCR-based method for detection and quantification of small RNAs. Biochem Biophys Res Commun 351(3): 756-63.
C. Chen, D. A. Ridzon, A. J. Broomer, Z. Zhou, D. H. Lee, J. T. Nguyen, M. Barbisin, N. L. Xu, V. R. Mahuvakar, M. R. Andersen, K. Q. Lao, K. J. Livak and K. J. Guegler. 2005. Real-time quantification of microRNAs by stem-loop RT-PCR. Nucleic Acids Res 33(20): e179.
F. Tang, P. Hajkova, S. C. Barton, K. Lao and M. A. Surani. 2006. MicroRNA expression profiling of single whole embryonic stem cells. Nucleic Acids Res 34(2): e9.

1. A method for detecting, quantifying, and/or profiling a target miRNA comprising:
a) contacting a sample containing miRNAs with an effective amount of poly(A)polymerase molecules to yield 3′ end-polyadenylated miRNA molecules,
b) contacting the sample with an effective amount of a universal primer for reverse transcription and reverse transcriptases, and reverse transcribing the polyadenylated miRNA molecules to yield corresponding c-DNA molecules, and
c) contacting the sample with an effective amount of a universal reverse primer and a forward primer, and amplifying the corresponding c-DNA molecules using an amplification reaction;
wherein the universal primer for reverse transcription is an oligonucleotide comprising: a (dT)ₙ sequence flanked by a stem-looped universal adaptor sequence, wherein “n” is an integer ranging from 10 to 50, wherein the universal primer comprises at least two nucleotides adjacent to the 3′ end of the (dT)ₙ sequence, and the nucleotide immediately adjacent to the (dT)ₙ sequence is not T, and wherein the universal adaptor sequence near the 5′ end of the (dT)ₙ sequence forms into a stem-loop structure by base-paring,
wherein the universal reverse primer in the amplification reaction is an oligonucleotide comprising a sequence that is, or base-pairs with, at least part of the adaptor sequence near the 5′ end of the (dT)ₙ sequence, and
wherein the forward primer in the amplification reaction is an oligonucleotide comprising a sequence that hybridizes, or base-pairs with, at least part of the target miRNA sequence.

2. The method, according to claim 1, wherein the amplification reaction for cDNA amplification is quantitative real time polymerase chain reaction (qRT-PCR).

3. The method, according to claim 1, comprising:
contacting the sample with a sufficient amount of a universal probe,
detecting and/or quantifying a level of amplified c-DNA molecules, and
determining the level of the target miRNA based on the c-DNA level;
wherein the universal probe comprises a sequence that is, or base-pairs with, at least part of the adaptor sequence near the 5′end of the (dT)ₙ sequence.

4. The method, according to claim 3, wherein the universal probe comprises a detectable moiety.

5. The method, according to claim 4, wherein the detectable moiety is a fluorophore selected from FAM, CY5, CY3, BODIPY FL, TEXAS RED, or any combinations of two or more of the foregoing.

6. The method, according to claim 1, wherein the amplification reaction for amplification of cDNA molecules is performed once.

7. The method, according to claim 1, wherein the threshold cycle (Ct) of the amplification reaction for amplification cDNA molecules ranges from 17 to 35.

8. The method, according to claim 1, wherein the sample is a total RNA sample.

9. The method, according to claim 1, wherein the universal primer for reverse transcription comprises SEQ ID NO: 1.

10. The method, according to claim 1, wherein the universal reverse primer in the amplification comprises SEQ ID NO: 2.

11. The method, according to claim 3, wherein the universal probe sequence comprises SEQ ID NO: 3.

12. The method, according to claim 1, wherein the method is capable of detecting, quantifying and/or profiling microRNA in a sample of about 1 pg.

13. A universal primer for detecting, quantifying and/or profiling miRNA, wherein the universal primer is a primer for reverse transcription of miRNAs, wherein the universal primer is an oligonucleotide comprising: a (dT)ₙ sequence flanked by a stem-looped universal adaptor sequence, wherein “n” is an integer ranging from 10 to 50, wherein the universal primer comprises at least two nucleotides adjacent to the 3′end of the (dT)ₙ sequence, and the nucleotide immediately adjacent to the (dT)ₙ sequence is not T, and wherein the universal adaptor sequence near the 5′end of the (dT)ₙ sequence forms into a stem-loop structure by base-paring.

14. The universal primer for reverse transcription according to claim 13, comprising SEQ ID NO: 1.

15. A universal primer for detecting, quantifying and/or profiling miRNA, wherein the universal primer is a reverse primer for an amplification reaction, wherein the universal primer is an oligonucleotide comprising a sequence that is, or base-pairs with, at least part of a universal adaptor sequence.

16. The universal reverse primer according to claim 15, comprising SEQ ID NO: 2.

17. A universal probe for detecting, quantifying and/or profiling miRNA, wherein the universal probe comprises a sequence that is, or base-pairs with, at least part of a universal adaptor sequence.

18. The universal probe according to claim 17, comprising a detectable moiety.

19. The universal probe according to claim 18, wherein the detectable moiety comprises a fluorophore.

20. A kit for detecting, quantifying and/or profiling a level of expression of miRNA, comprising a universal primer for reverse transcription and a universal reverse primer for an amplification reaction.

21. The kit according to claim 20, further comprising a universal probe.


SEQUENCE LISTING (9 sequences)

SEQ ID NO: 1 - DNA, 44 bp, Artificial Sequence
  RTUloop (universal primer)
tggctagttaagctcaccagctcggtaccaagcttaactagcca

SEQ ID NO: 2 - DNA, 24 bp, Artificial Sequence
  QRTU (universal reverse primer)
tggctagttaagctcaccagctcg

SEQ ID NO: 3 - DNA, 26 bp, Artificial Sequence
  miRU probe
tggctagttaagcttggtaccgagct

SEQ ID NO: 4 - DNA, 22 bp, Artificial Sequence
  Let-7a (forward primer)
tgaggtagtaggttgtatagtt

SEQ ID NO: 5 - DNA, 22 bp, Artificial Sequence
  miR-21 (forward primer)
tagcttatcagactgatgttga

SEQ ID NO: 6 - DNA, 23 bp, Artificial Sequence
  miR-142 (forward primer)
tgtagtgtttcctactttatgga

SEQ ID NO: 7 - DNA, 22 bp, Artificial Sequence
  miR-150 (forward primer)
tctcccaacccttgtaccagtg

SEQ ID NO: 8 - DNA, 24 bp, Artificial Sequence
  miR-494 (forward primer)
tgaaacatacacgggaaacctctt

SEQ ID NO: 9 - DNA, 22 bp, Artificial Sequence
  mmu-miR-690 (forward primer)
aaaggctaggctcacaaccaaa